(19) United States
(12) Patent Application Publication
(10) Pub. No.: US 2025/0241993 A1
(43) Pub. Date: Jul. 31, 2025

(54) MIXED PROTOCOL FOR TREATMENT OF INFERTILITY

(71) Applicant: Ferring B.V., Hoofddorp (NL)

(72) Inventors: Francois BISSONNETTE, Montreal (CA); Isaac-Jacques KADOCH, Montreal (CA); Albert YUZPE, Vancouver (CA)

(73) Assignee: Ferring B.V., Hoofddorp (NL)

(21) Appl. No.: 18/853,024

(22) PCT Filed: Apr. 1, 2022
(86) PCT No.: PCT/EP2022/058805

§ 371 Date: Sep. 30, 2024

(51) Int. Cl. A61K 38/24; A61K 9/00; A61P 15/08

(52) U.S. Cl. CPC A61K 38/24; A61K 9/0019; A61P 15/08

(57) ABSTRACT

Methods, uses and products including a recombinant human follicle stimulating hormone (rFSH) including α2,3- and α2,6-sialylation and menotropin (human menopausal gonadotropin or hMG) for use in the treatment of infertility in a patient of age≥35 are described.

The present invention relates to pharmaceutical products and methods for the treatment of infertility.

### BACKGROUND

Assisted reproductive technologies (ART) such as in vitro fertilisation (IVF) are well known. ART generally requires a step of controlled ovarian stimulation (COS), in which a cohort of follicles is stimulated to full maturity. Standard COS regimens include administration of gonadotrophins, such as menotropin (e.g., human menopausal gonadotropin (hMG)) and follicle stimulating hormone (FSH) alone or in combination with luteinising hormone (LH) activity to stimulate multiple follicular development. Usually COS requires administration of a GnRH analogue (GnRH agonist) or GnRH antagonist prior to and/or during stimulation to prevent a premature LH surge which may induce ovulation before planned oocyte retrieval. The aim of COS is generally to stimulate a number of follicles to maturity so, when ovulation is triggered, a number of oocytes are retrieved for fertilization. In general, the best oocytes are selected for implantation while others may be cryopreserved for future use.

In case of a too high ovarian response, COS can be associated with a risk of ovarian hyperstimulation syndrome (OHSS), which can lead to cancellation of the COS cycle and can become life threatening in severe cases. The ability to predict the ovarian response potential of women to COS may allow the development of personalised or individualised COS protocols. Such individualised protocols could, for example, reduce the risk of OHSS in women predicted to have an excessive ovarian response to COS, and prevent cancellation of COS cycles. Levels of anti-Müllerian hormone (AMH) are directly correlated with the ovarian response to gonadotrophins during COS. Thus, high levels of AMH are a good predictor of excessive ovarian response, and an indicator of risk of OHSS, whereas low levels of AMH predict a poor ovarian response to COS. Other predictors of ovarian response include age, body mass index (BMI), FSH, and antral follicle count (AFC).

In humans, FSH and LH work in concert to stimulate folliculogenesis and ovulation. Therefore, these gonadotropins are typically used for COS in order to increase the number of oocytes produced in IVF. This FSH—LH combination concept has led to so-called “mixed protocols” of concomitant treatment with hMG and FSH for ovarian stimulation being widely used. Indeed, COS protocols that utilize both hMG (a source of both FSH and LH activity) and FSH are commonly used in an attempt to obtain better quality oocytes and embryos and thus, higher pregnancy rates compared to the use of recombinant FSH alone. Determination of a stimulation protocol and FSH dosage typically is established by the physician on the basis of various parameters, which may include various combinations of the patient's age, body weight and ovarian reserve profile (AMH, AFC, BMI etc.). However, to date a mixed protocol of follitropin delta (a recombinant FSH expressed in a human cell line) and HP-hMG has not been individualised for patients over the age of 35.

Thus, there is a need for improved assisted reproductive technology methods, particularly improved mixed hMG and FSH protocols for patients over the age of 35.

### SUMMARY

In the exploratory study described below, the applicants demonstrated the effectiveness of a mixed protocol of individualised dosing of a menotropin (human menopausal gonadotropin (hMG)) and a recombinant human follicle stimulating hormone (rFSH) including α2,3-and α2,6-sialylation (e.g., follitropin delta) in the treatment of infertility in patients of age≥35 based on AMH and bodyweight.

The results of the study demonstrated that the number of good-quality, usable blastocysts obtained by day 5 or 6 of embryo culture can be statistically significantly increased by using this mixed protocol without compromising safety, compared with cycles where follitropin delta was used as monotherapy. The subgroup of 35- to 36-year-old and 37- to 40-year-old patients are the ones that appear to benefit the most from the application of this combination regimen. For example, the mean (adjusted) number of good-quality blastocysts on day 5-6 after fertilisation was 4.03±0.72 and 4.24±0.62 for the 35.0-36.9 and 37.0-40.0 age group respectively compared to 2.0±2.2 (for all age groups) where follitropin delta was used as monotherapy. The incidence of any grade of OHSS was 9.3% (7.4% for the older age groups, 35.0-36.9 and 37.0-40.0) compared to 2.6% where follitropin delta was used as monotherapy. No cases of moderate or severe OHSS were observed in the study compared with cases in 1.4% patients where follitropin delta was used as monotherapy. Thus, the dosing regimen disclosed herein achieves the desired goal. The percentage of patients who did not require dose adjustment of menotropin (HP-hMG) was 70.83% and 54.84% for the 35.0-36.9 and 37.0-40.0 age group respectively. As the study was exploratory, adjustments in the medication regime were expected, therefore it is remarkable that no change in HP-hMG dose was required for this percentage of these patients.

In accordance with some aspects, there are provided products comprising a menotropin (human menopausal gonadotropin (hMG), such as highly purified hMG (HP-hMG)) and a recombinant human follicle stimulating hormone (rFSH) including α2,3- and α2,6-sialylation; wherein the product is for use in the treatment of infertility in a patient of age≥35 (e.g., age 35 to 40, e.g., age 35-36) having a serum AMH level of <15 pmol/L prior to treatment/stimulation; wherein the product is to be administered at a dose of, or equivalent to, 11-13 μg (e.g., 12 μg) per day of the recombinant human FSH including α2,3- and α2,6-sialylation, and a dose of, or equivalent to, 37.5 IU to 225 IU HP-hMG per day of the menotropin. The dose of hMG may be a dose of, or equivalent to, 75 IU to 225 IU HP-hMG per day of the menotropin.

The use may comprise a step of determining the serum AMH level of the patient, and a step of administering the dose of recombinant human FSH including α2,3- and α2,6-sialylation and the dose of menotropin to a patient having a serum AMH level of <15 pmol/L. The use may further comprise a step of determining the serum estradiol (E₂) level of the patient during treatment/stimulation (e.g., to monitor the response to stimulation and subsequently adjust the menotropin dose or e.g., to assist in the choice of trigger medication).

In some aspects, the product may be administered from day 1 of stimulation to at least day 5, 6 or 7 of stimulation. From day 6, 7 or 8 of stimulation, the product may be administered (with the dose of rFSH maintained at the (starting) day 1 dose) at a dose per day of menotropin which is maintained, increased or decreased based on the serum estradiol (E₂) level of the patient on day 5, 6 or 7 of stimulation. From day 6, 7 or 8 of stimulation, the dose per day of menotropin may be (i) decreased (relative to the starting dose) by up to 75 IU (for example decreased by 37.5 IU, decreased by 75 IU, or decreased by any amount in between 37.5 IU and 75 IU) in a patient having an estradiol (E₂) level of >3000 pmol/L on day 5, 6 or 7 of stimulation; or (ii) decreased to zero in a patient having an estradiol (E₂) level of >3000 pmol/L on day 5, 6 or 7 of stimulation. From day 6, 7 or 8 of stimulation, the dose per day of menotropin may be (i) increased (relative to the starting dose) by up to 150 IU (for example increased by 75 IU, increased by 150 IU, or increased by any amount in between 75 IU and 150 IU) in a patient having an estradiol (E₂) level of 750-999 pmol/L on day 5, 6 or 7 of stimulation or (ii) increased to a dose of 225 IU in a patient having an estradiol (E₂) level of <750 pmol/L on day 5, 6 or 7 of stimulation. From day 6, 7 or 8 of stimulation the dose per day of menotropin may be maintained (at the starting dose) in a patient having an estradiol (E₂) level of between 1000 and 3000 pmol/L on day 5, 6 or 7 of stimulation.

The use may comprise a further step of triggering ovulation by administering hCG to a patient who, following stimulation, has ≥3 follicles of ≥17 mm in diameter each and estradiol (E₂) levels <10,000 pmol/L; or administering a GnRH agonist to a patient who, following stimulation, has ≥3 follicles of ≥17 mm in diameter each and estradiol (E₂) levels between 10,000 pmol/L and 15,000 pmol/L; or administering a GnRH agonist to a patient who, following stimulation, has ≥20 follicles of ≥12 mm in diameter each and/or estradiol (E₂) levels ≥15,000 pmol/mL or ≥20,000 pmol/mL. The dose of hCG to be administered to the patient may be from 5,000 IU to 10,000 IU.

The use may comprise a further step of administering a GnRH antagonist starting on day 6 of stimulation.

The use may further comprise one or more or all of: retrieving (e.g. harvesting) oocyte(s); fertilizing (e.g. inseminating) the oocytes(s); allowing the fertilized oocytes to develop to the blastocyst stage; and freezing one or more (or all) blastocysts.

In some aspects, the menotropin and a rFSH including α2,3- and α2,6-sialylation are administered separately, for example the rFSH including α2,3- and α2,6-sialylation is for administration in the morning and the menotropin is for administration in the afternoon or evening. In some aspects, the menotropin and a rFSH including α2,3- and α2,6-sialylation are administered together.

In some aspects, the menotropin is highly purified menotropin (HP-hMG). In some aspects, the rFSH is an rFSH wherein 50 to 95% of the total sialylation is α2,3-sialylation and wherein 5-50% of the total sialylation is α2,6-sialylation. In some aspects, the rFSH is an rFSH wherein 80 to 90% of the total sialylation is α2,3-sialylation and wherein 10-20% of the total sialylation is α2,6-sialylation (e.g., follitropin delta, available from Ferring B. V.).

In some aspects, the initial daily dose of menotropin (hMG) is: 75 IU when the recombinant human follicle stimulating hormone (rFSH) including α2,3- and α2,6-sialylation daily dose is <12 μg; 150 IU when the recombinant human follicle stimulating hormone (rFSH) including α2,3- and α2,6-sialylation daily dose is 12 μg and patient body weight is <100 kg; and 225 IU when the recombinant human follicle stimulating hormone (rFSH) including α2,3- and α2,6-sialylation daily dose is 12 μg and patient body weight is ≥100 kg.

The use may comprise a further step of assessing follicular growth by ultrasonography e.g., on day 9, 10, 11, 12, 13, 14 etc. of stimulation.

In accordance with some aspects, there are provided products comprising a menotropin and a recombinant human follicle stimulating hormone (rFSH) including α2,3- and α2,6-sialylation; wherein the product is for use in the treatment of infertility in a patient of age≥35 (e.g., age 35 to 40, e.g. age 35-36) having a serum AMH level of ≥15 pmol/L (e.g., 15-35 pmol/L) prior to treatment/stimulation; and wherein the product is to be administered at a dose of, or equivalent to, 0.09-0.19 μg recombinant human FSH including α2,3- and α2,6-sialylation per kg body weight of the patient per day, and a dose of, or equivalent to, 37.5 IU to 225 IU HP-hMG per day of the menotropin. The dose of hMG may be a dose of, or equivalent to, 75 IU to 225 IU HP-hMG per day of the menotropin.

The use may comprise a step of determining the serum AMH level of the patient, and a step of administering the dose of recombinant human FSH including α2,3- and α2,6-sialylation and the dose of menotropin to a patient having a serum AMH level of ≥15 pmol/L (e.g., 15-35 pmol/L). The use may further comprise a step of determining the serum estradiol (E₂) level of the patient during treatment/stimulation (e.g., to monitor the response to stimulation and subsequently adjust the menotropin dose or e.g., to assist in the choice of trigger medication).

In some aspects, the product may be administered from day 1 of stimulation to at least day 5, 6 or 7 of stimulation. From day 6, 7 or 8 of stimulation, the product may be administered (with the dose of rFSH maintained at the (starting) day 1 dose) at a dose per day of menotropin which is maintained, increased or decreased based on the serum estradiol (E₂) level of the patient on day 5, 6 or 7 of stimulation. From day 6, 7 or 8 of stimulation, the dose per day of menotropin may be (i) decreased (relative to the starting dose) by up to 75 IU (for example decreased by 37.5 IU, decreased by 75 IU, or decreased by any amount in between 37.5 IU and 75 IU) in a patient having an estradiol (E₂) level of >3000 pmol/L on day 5, 6 or 7 of stimulation; or (ii) decreased to zero in a patient having an estradiol (E₂) level of >3000 pmol/L on day 5, 6 or 7 of stimulation. From day 6, 7 or 8 of stimulation, the dose per day of menotropin may be (i) increased (relative to the starting dose) by up to 150 IU (for example increased by 75 IU, increased by 150 IU, or increased by any amount in between 75 IU and 150 IU) in a patient having an estradiol (E₂) level of 750-999 pmol/L on day 5, 6 or 7 of stimulation or (ii) increased to a dose of 225 IU in a patient having an estradiol (E₂) level of <750 pmol/L on day 5, 6 or 7 of stimulation. From day 6, 7 or 8 of stimulation the dose per day of menotropin may be maintained (at the starting dose) in a patient having an estradiol (E₂) level of between 1000 and 3000 pmol/L on day 5, 6 or 7 of stimulation.

The use may comprise a further step of triggering ovulation by administering hCG to a patient who, following stimulation, has ≥3 follicles of ≥17 mm in diameter each and estradiol (E₂) levels <10,000 pmol/L; or administering a GnRH agonist to a patient who, following stimulation, has ≥3 follicles of ≥17 mm in diameter each and estradiol (E₂) levels between 10,000 pmol/L and 15,000 pmol/L; or administering a GnRH agonist to a patient who, following stimulation, has ≥20 follicles of ≥12 mm in diameter each and/or estradiol (E₂) levels ≥15,000 pmol/mL or ≥20,000 pmol/mL. The dose of hCG to be administered to the patient may be from 5,000 IU to 10,000 IU.

The use may comprise a further step of administering a GnRH antagonist starting on day 6 of stimulation.

The use may further comprise one or more or all of: retrieving (e.g. harvesting) oocyte(s); fertilizing (e.g. inseminating) the oocytes(s); allowing the fertilized oocytes to develop to the blastocyst stage; and freezing one or more (or all) blastocysts.

In some aspects, the menotropin and a rFSH including α2,3- and α2,6-sialylation are administered separately, for example the rFSH including α2,3- and α2,6-sialylation is for administration in the morning and the menotropin is for administration in the afternoon or evening. In some aspects, the menotropin and a rFSH including α2,3- and α2,6-sialylation are administered together.

In some aspects, the menotropin is highly purified menotropin (HP-hMG). In some aspects, the rFSH is an rFSH wherein 50 to 95% of the total sialylation is α2,3-sialylation and wherein 5-50% of the total sialylation is α2,6-sialylation. In some aspects, the rFSH is an rFSH wherein 80 to 90% of the total sialylation is α2,3-sialylation and wherein 10-20% of the total sialylation is α2,6-sialylation (e.g., follitropin delta, available from Ferring B. V.).

In some aspects, the initial daily dose of menotropin (hMG) is: 75 IU when the recombinant human follicle stimulating hormone (rFSH) including α2,3- and α2,6-sialylation daily dose is <12 μg; 150 IU when the recombinant human follicle stimulating hormone (rFSH) including α2,3- and α2,6-sialylation daily dose is 12 μg and patient body weight is <100 kg; and 225 IU when the recombinant human follicle stimulating hormone (rFSH) including α2,3- and α2,6-sialylation daily dose is 12 μg and patient body weight is ≥100 kg.

The use may comprise a further step of assessing follicular growth by ultrasonography e.g., on day 9, 10, 11, 12, 13, 14 etc. of stimulation.

In accordance with some aspects, there is provided a method of treatment of infertility in a patient of age≥35 (e.g., age 35 to 40, e.g., age 35-36) and having a serum AMH level of <15 pmol/L prior to treatment, comprising; identifying a patient of age≥35 (e.g., age 35 to 40, e.g., age 35-36) and having a serum AMH level of <15 pmol/L prior to treatment; and administering to the patient human menopausal gonadotropin (hMG) and a recombinant human follicle stimulating hormone (rFSH) including α2,3- and α2,6-sialylation, wherein the rFSH is administered at a dose a dose of, or equivalent to, 11-13 μg (e.g., 12 μg) per day of the rFSH, and the hMG is administered at a dose of, or equivalent to, 37.5 IU to 225 IU per day of highly purified hMH (HP-hMG). The dose of hMG may be a dose of, or equivalent to, 75 IU to 225 IU HP-hMG per day of the menotropin.

In accordance with some aspects, there is provided a method of treatment of infertility in a patient of age≥35 (e.g., age 35 to 40, e.g., age 35-36) and having a serum AMH level of <15 pmol/L prior to treatment/, comprising; identifying a patient of age≥35 (e.g., age 35 to 40, e.g., age 35-36) and having a serum AMH level of ≥15 pmol/L (e.g., 15-35 pmol/L) prior to treatment; and administering to the patient human menopausal gonadotropin (hMG) and a recombinant human follicle stimulating hormone (rFSH) including α2,3- and α2,6-sialylation, wherein the rFSH is administered at a dose a dose of, or equivalent to, 0.09-0.19 μg of the rFSH per kg body weight of the patient per day, and the hMG is administered at a dose of, or equivalent to, 37.5 IU to 225 IU per day of highly purified hMG (HP-hMG). The dose of hMG may be a dose of, or equivalent to, 75 IU to 225 IU HP-hMG per day of the menotropin.

In accordance with some aspects, there is provided a pharmaceutical composition comprising a menotropin (human menopausal gonadotropin or hMG) for use in the treatment of infertility in a patient of age≥35 (e.g., age 35 to 40, e.g., age 35-36) having a serum AMH level of <15 pmol/L prior to treatment/stimulation; wherein the pharmaceutical composition is to be administered at a dose of, or equivalent to, 37.5 IU to 225 IU HP-hMG per day of the menotropin, together with a dose of, or equivalent to, 11-13 μg (e.g., 12 μg) per day of a recombinant human FSH (rFSH) including α2,3- and α2,6-sialylation.

In accordance with some aspects, there is provided a pharmaceutical composition comprising a recombinant human follicle stimulating hormone (rFSH) including α2,3- and α2,6-sialylation for use in the treatment of infertility in a patient of age≥35 (e.g., age 35 to 40, e.g., age 35-36) having a serum AMH level of <15 pmol/L prior to treatment/stimulation; wherein the pharmaceutical composition is to be administered at a dose of, or equivalent to, 11-13 μg (e.g., 12 μg) per day of the recombinant human FSH including α2,3- and α2,6-sialylation, together with a dose of, or equivalent to, 37.5 IU to 225 IU HP-hMG per day of a menotropin (human menopausal gonadotropin or hMG).

In accordance with some aspects, there is provided a pharmaceutical composition comprising a menotropin (human menopausal gonadotropin or hMG) for use in the treatment of infertility in a patient of age≥35 (e.g., age 35 to 40, e.g., age 35-36) having a serum AMH level of ≥15 pmol/L (e.g., 15-35 pmol/L) prior to treatment/stimulation; wherein the pharmaceutical composition is to be administered at a dose of, or equivalent to, 37.5 IU to 225 IU HP-hMG per day of the menotropin, together with a dose of, or equivalent to, 0.09-0.19 μg per kg body weight of the patient per day of a recombinant human FSH including α2,3- and α2,6-sialylation.

In accordance with some aspects, there is provided a pharmaceutical composition comprising a recombinant human follicle stimulating hormone (rFSH) including α2,3- and α2,6-sialylation for use in the treatment of infertility in a patient of age≥35 (e.g., age 35 to 40, e.g., age 35-36) having a serum AMH level of ≥15 pmol/L (e.g., 15-35 pmol/L) prior to treatment/stimulation; wherein the pharmaceutical composition is to be administered at a dose of, or equivalent to, 0.09-0.19 μg per kg body weight of the patient per day of the recombinant human FSH including α2,3- and α2,6-sialylation, together with a dose of, or equivalent to, 37.5 IU to 225 IU HP-hMG per day of the menotropin (human menopausal gonadotropin or hMG).

### DETAILED DESCRIPTION

The present applicants have demonstrated the effectiveness of a mixed protocol of individualised dosing of a menotropin (hMG) and a recombinant human follicle stimulating hormone (rFSH) including α2,3- and α2,6-sialylation (e.g., follitropin delta) in the treatment of infertility in patients of age≥35 based on AMH and bodyweight.

The results of the study demonstrated that the number of good-quality, usable blastocysts obtained by day 5 or 6 of embryo culture can be statistically significantly increased by using this mixed protocol without compromising safety, compared with cycles where follitropin delta was used as monotherapy. The subgroup of 35- to 36-year-old and 37- to 40-year-old patients are the ones that appear to benefit the most from the application of this combination regimen. For example, the mean (adjusted) number of good-quality blastocysts on day 5-6 after fertilisation was 4.03±0.72 and 4.24±0.62 for the 35.0-36.9 and 37.0-40.0 age group respectively compared to 2.0±2.2 (for all age groups) where follitropin delta was used as monotherapy. The incidence of any grade of OHSS was 9.3% (7.4% for the older age groups, 35.0-36.9 and 37.0-40.0) compared to 2.6% where follitropin delta was used as monotherapy. No cases of moderate or severe OHSS were observed in the study compared with cases in 1.4% of patients where follitropin delta was used as monotherapy. Thus, the dosing regimen disclosed herein achieves the desired goal. The percentage of patients who did not require dose adjustment of menotropin (HP-hMG) was 70.83% and 54.84% for the 35.0-36.9 and 37.0-40.0 age group respectively. As the study was exploratory, adjustments in the medication regime were expected, therefore it is remarkable that no change in HP-hMG dose was required for this percentage of these patients.

Therefore, provided herein are individualised treatments and dosing regimens constructed for use to treat a patient of age≥35 for infertility.

### Definitions

Technical and scientific terms used herein have the meanings commonly understood by one of ordinary skill in the art of assisted reproductive technology to which the present invention pertains, unless otherwise defined. Reference is made herein to various methodologies known to those of ordinary skill in the art. Any suitable materials and/or methods known to those of ordinary skill in the art can be utilized in carrying out the present invention based on the guidance provided herein. However, specific materials and methods are described. Materials, reagents and the like to which reference is made in the following description and examples are obtainable from commercial sources, unless otherwise noted.

It is to be understood, that any definitions and terms herein defined are meant to have the same meaning and purpose in any of the aspects and embodiments of the invention unless explicitly otherwise stated not to.

As used herein, the singular forms “a,” “an,” and “the” designate both the singular and the plural, unless expressly stated to designate the singular only.

As used herein, the term “about” means that the number or range so modified is not limited to the exact number or range set forth, but encompass ranges around the stated number or range as will be understood by persons of ordinary skill in the art depending on the context in which the number or range is used. Unless otherwise apparent from the context or convention in the art, “about” means up to plus or minus 10% of the particular term.

As used herein “ongoing pregnancy” refers to pregnancy with a viable foetus and detectable foetal heartbeat at 10-11 weeks gestation, e.g., at 8-9 weeks post blastocyst/embryo transfer.

As used herein “clinical pregnancy” refers to gestation and a detectable foetal heartbeat at 5-6 weeks gestation, e.g., at 3-4 weeks post blastocyst/embryo transfer.

Herein the terms “patient” and “subject” and “female” and “woman” are used interchangeably.

The term “treatment of infertility” includes treatment of infertility in a subject having tubal or unexplained infertility, including treatment of infertility in a subject having endometriosis, for example stage I or stage II endometriosis, and/or in a subject with a partner with male factor infertility. The product may be for (use in) the treatment of infertility (and/or for controlled ovarian stimulation) in a subject having endometriosis, for example in a subject having stage I or stage II endometriosis, as defined by The American Society for Reproductive Medicine (ASRM) classification system for the various stages of endometriosis, (stage IV most severe; stage I least severe) [American Society for Reproductive Medicine. Revised American Society for Reproductive Medicine classification of endometriosis: 1996. Fertil Steril 1997; 67,817 821.].

The serum concentration of anti-Müllerian hormone (AMH) is now established as a reliable marker of ovarian reserve. Decreasing levels of AMH are correlated with reduced ovarian response to gonadotrophins during COS. Further, high levels of AMH are a good predictor of excessive ovarian response, and an indicator of risk of OHSS. In accordance with the present disclosure, for the first (and in some cases subsequent) treatment cycle, the individual daily dose of recombinant human follicle stimulating hormone (rFSH) including α2,3- and α2,6-sialylation is determined on the basis of the woman's serum AMH concentration and, in some embodiments, her body weight. The recombinant human follicle stimulating hormone (rFSH) including α2,3- and α2,6-sialylation dose is based on a recent determination of AMH (i.e. within the last 12 months), for example measured by the ELECSYS® AMH Plus immunoassay (Roche), or similar assays such as ACCESS AMH Advanced from Beckman Coulter or UMIPULSE G AMH from Fujirebio. Additionally or alternatively, the serum concentration of anti-Müllerian hormone (AMH) in the patient may be determined using a Beckmann-Coulter Gen 2 assay as described in Arce et al., Fertility and Sterility 99: 1644-53 (2013), or an equivalent AMH level assessed by a different method.

A subject may have a BMI>15 and BMI<40 kg/m², for example a BMI>17.5 and BMI<38 kg/m², for example a BMI>18 and BMI<25 kg/m², for example a BMI>20 and BMI<25 kg/m². Thus a product or method as described herein may be used for the treatment of infertility in a patient having BMI>15 and BMI<40 kg/m², for example a subject having BMI>17.5 and BMI<38 kg/m², for example a subject having BMI>18 and BMI<25 kg/m², for example a subject having BMI>20 and BMI<25 kg/m². Thus a product or method as described herein may be used for the treatment of infertility in a patient having BMI>17.5 and BMI<32 kg/m². BMI may be measured by methods well known in the art to identify the patient for treatment.

The term “follicle” herein means an ovarian follicle which is a fluid-filled sac that contains an immature egg, or oocyte.

A blastocyst forms in the early development of a human (or other mammal). In humans, blastocyst formation begins about 5 days after fertilization. The use of blastocysts in (IVF) generally involves retrieval (harvesting) from the woman a number of oocytes resulting from a controlled ovarian stimulation cycle; fertilization (insemination of) one or more oocytes and culturing the fertilized egg (oocyte) for five days to form a blastocyst (i.e. allowing the fertilized oocyte to develop to the blastocyst stage); and implanting the blastocyst into the uterus. An embryo forms in the early development of a human (or other mammal). The use of embryos in (IVF) generally involves retrieval (harvesting) from the woman a number of oocytes resulting from a controlled ovarian stimulation cycle; fertilization (insemination of) one or more oocytes and culturing the fertilized egg (oocyte) for e.g., 3 days to form an embryo (i.e. allowing the fertilized oocyte to develop to the embryo stage); and implanting the embryo into the uterus.

The term “menotropin” as used herein includes human menopausal gonadotropin (hMG). The term “highly purified menotropin” (HP-hMG) as used herein includes HP-hMG available under the trademark MENOPUR® from Ferring B. V., that contains both follicle stimulating hormone (FSH) and human chorionic gonadotropin (hCG)-driven luteinizing hormone (LH)-activity. In some examples, the hMG is a HP-hMG which includes both follicle stimulating hormone (FSH) and human chorionic gonadotropin (hCG)-driven luteinizing hormone (LH)-activity (e.g. MENOPUR®).

The term “GnRH agonist” as used herein includes gonadotropin-releasing hormone (GnRH) agonists such as buserelin (e.g., Suprecur®), leuprorelin (leuprolide acetate, e.g., Lupron®), nafarelin (e.g., Synarel®), and triptorelin (e.g., Trelstar®).

The term “GnRH antagonist” as used herein includes gonadotropin-releasing hormone (GnRH) antagonists, such as ganirelix acetate (e.g., Orgalutran®) and cetrorelix acetate (e.g., Cetrotide®), which block the action of GnRH by competitive blocking of the GnRH receptors on pituitary gonadotropes, and thus prevent gonadotropin production/release and premature ovulation (release of eggs).

In accordance with all aspects described herein, the recombinant human follicle stimulating hormone (rFSH) including α2,3- and α2,6-sialylation may be human cell line-derived recombinant FSH as described in more detail below. In all aspects, the recombinant FSH including α2,3- and α2,6-sialylation may be that sold under the trademark REKOVELLE® (follitropin delta) (Ferring B. V.). In all aspects, the recombinant FSH may be administered by injection, e.g., subcutaneous injection.

As used herein, “day one of treatment”, also referred to as “day one of stimulation”, refers to the first day that the dose of menotropin and recombinant human FSH including α2,3- and α2,6-sialylation is administered to the patient. Day one of treatment (stimulation) may take place on day 1, 2 or 3, for example on day 2 or day 3, of the patient's menstrual cycle. In other words, day one of treatment (stimulation) may be one, two or three days, for example two or three days, after the patient commences menstrual bleeding, consistent with usage of this term in clinical practice with GnRH antagonist or GnRH agonist protocols. The term “during treatment” means on a day or on days that menotropin and recombinant human FSH including α2,3- and α2,6-sialylation is being administered to the patient.

In the treatments, methods and uses described herein, the administration of menotropin and recombinant human FSH including α2,3- and α2,6-sialylation starts on day one of treatment and may continue for two to twenty days, for example continue for 2, 3, 4, 5, 6, 7, 8, 9, 10, 11, 12, 13, 14, 15, 16, 17, 18, 19, or 20 days. The administration of menotropin and recombinant FSH starts on day one of treatment and may continue for four to twenty days, for example seven to thirteen days, for example nine to thirteen days, for example 10 to 13 days, for example 10 to 11 days. The dose of recombinant FSH is the same every day, as determined by an established algorithm based on body weight and serum AMH level as set out below. However, the daily dose of menotropin may be adjusted from the mid-follicular stage of stimulation depending on the patient's serum estradiol (E₂) level measured at the mid-follicular stage.

It will be appreciated that the mid-follicular stage of stimulation occurs typically after 4 to 6 days of stimulation, such as on the 5^{th}, 6^{th} or 7^{th} day of stimulation. Therefore, the patient's serum estradiol (E₂) level may be measured during mid-follicular stage of stimulation (e.g., day 5, 6 or 7). Adjustment of the dose per day of menotropin may be made from the day after the day on which the patient's serum estradiol (E₂) level was measured. Therefore, depending on whether the patient's serum estradiol (E₂) level was measured on day 5, 6 or day 7 of stimulation; the dose per day of menotropin may be adjusted (increased or decreased) or maintained from day 6, 7 or 8 of stimulation.

As used herein “equivalent to” in relation to dosing means a dose that has the same pharmaceutical effect as the dose recited in the claim or herein. For example a dose of, or equivalent to, 12 μg per day may be the recited dose of 12 μg per day or may be a pharmaceutically equivalent dose such as a dose of 36 μg every 3 days. It will be appreciated this applies to any dose recited herein.

### Treatments

In the exploratory study described below, the inventors demonstrated the effectiveness of a mixed protocol of individualised dosing of a menotropin (hMG, e.g., HP-hMG) and a recombinant human follicle stimulating hormone (rFSH) including α2,3- and α2,6-sialylation (e.g., follitropin delta) in the treatment of infertility in patients of age≥35 based on AMH and bodyweight.

The results of the study demonstrated that the number of good-quality, usable blastocysts obtained by day 5 or 6 of embryo culture can be statistically significantly increased by using this mixed protocol without compromising safety, compared with cycles where follitropin delta was used as monotherapy. The subgroup of 35- to 36-year-old and 37- to 40-year-old patients are the ones that appear to benefit the most from the application of this combination regimen. For example, the mean (adjusted) number of good-quality blastocysts on day 5-6 after fertilisation was 4.03±0.72 and 4.24±0.62 for the 35.0-36.9 and 37.0-40.0 age group respectively compared to 2.0±2.2 (for all age groups) where follitropin delta was used as monotherapy. The incidence of any grade of OHSS was 9.3% (7.4% for the older age groups, 35.0-36.9 and 37.0-40.0) compared to 2.6% where follitropin delta was used as monotherapy. No cases of moderate or severe OHSS were observed in the study compared with cases in 1.4% patients where follitropin delta was used as monotherapy. The percentage of patients who did not require dose adjustment of menotropin (HP-hMG) was 70.83% and 54.84% for the 35.0-36.9 and 37.0-40.0 age group respectively. As the study was exploratory, adjustments in the medication regime were expected, therefore it is remarkable that no change in HP-hMG dose was required for this percentage of these patients.

In a first aspect there is provided a product comprising a menotropin (hMG) and a recombinant human follicle stimulating hormone (rFSH) including α2,3- and α2,6-sialylation; wherein the product is for use in the treatment of infertility in a patient of age≥35 (e.g., age 35 to 40, e.g., age 35-36) having a serum AMH level of <15 pmol/L prior to treatment/stimulation; wherein the product is to be administered at a dose of, or equivalent to, 11-13 μg (e.g., 12 μg) per day of the recombinant human FSH including α2,3- and α2,6-sialylation, and a dose of, or equivalent to, 37.5 IU to 225 IU HP-hMG per day of the menotropin. The dose of hMG may be a dose of, or equivalent to, 75 IU to 225 IU HP-hMG per day of the menotropin.

The use may comprise a step of determining the serum AMH level of the patient (e.g. using the automated Elecsys® AMH assay from Roche Diagnostics or similar), and a step of administering the dose of recombinant human FSH including α2,3- and α2,6-sialylation and the dose of menotropin to a patient having a serum AMH level of <15 pmol/L.

The patient may be of age 35 to 40 years or of age>40 years. The use may comprise a step of determining the age of the patient, and a step of administering a menotropin (hMG) and a recombinant human follicle stimulating hormone (rFSH) including α2,3- and α2,6-sialylation a patient of age 35-40 years, for example 35-36 years. The use may comprise a step of determining the age of the patient, and a step of administering a menotropin (hMG) and a recombinant human follicle stimulating hormone (rFSH) including α2,3- and α2,6-sialylation to a patient of age>40 years.

The use may further comprise a step or steps of determining the serum estradiol (E₂) level of the patient during treatment/stimulation. The serum estradiol (E₂) level of the patient may be determined for the purposes of monitoring the response to stimulation and subsequently adjusting the dose per day dose of menotropin and/or for the choice of triggering medication for final follicular maturation as described in more detail below. The serum estradiol (E₂) levels may be determined via methods known in the art.

The product may be administered from day 1 of stimulation to at least day 5, 6 or 7 of stimulation. From day 6, 7 or 8 of stimulation, the product may be administered (with the dose of rFSH maintained at the (starting) day 1 dose) at a dose per day of menotropin which is maintained, increased or decreased based on the serum estradiol (E₂) level of the patient on day 5, 6 or 7 of stimulation. Thus, the use may comprise a step of determining the serum estradiol (E₂) level of the patient on day 5, 6 or 7, and a step of administering a dose per day of menotropin (human menopausal gonadotropin or hMG) which is maintained, increased or decreased relative to the starting dose to a patient from day 6, 7 or 8 of stimulation.

From day 6, 7 or 8 of stimulation, the dose per day of menotropin may be (i) decreased (relative to the starting dose) by up to 75 IU (for example decreased by 37.5 IU, decreased by 75 IU, or any amount in between 37.5 IU and 75 IU) in a patient having an estradiol (E₂) level of >3000 pmol/L on day 5, 6 or 7 of stimulation; or (ii) decreased to zero in a patient having an estradiol (E₂) level of >3000 pmol/L on day 5, 6 or 7 of stimulation. Thus, the use may comprise a step of determining the serum estradiol (E₂) level of the patient on day 5, 6 or 7, and a step of administering a dose per day of menotropin (hMG) which is (i) decreased (relative to the starting dose) by up to 75 IU (for example decreased by 37.5 IU, decreased by 75 IU, or any amount in between 37.5 IU and 75 IU) to a patient having an estradiol (E₂) level of >3000 pmol/L on day 5, 6 or 7 of stimulation from day 6, 7 or 8 of stimulation; or (ii) decreased to zero in a patient having an estradiol (E₂) level of >3000 pmol/L on day 5, 6 or 7 of stimulation from day 6, 7 or 8 of stimulation.

From day 6, 7 or 8 of stimulation, the dose per day of menotropin may be (i) increased (relative to the starting dose) by up to 150 IU (for example increased by 75 IU, increased by 150 IU, or any amount in between 75 IU and 150 IU) in a patient having an estradiol (E₂) level of 750-999 pmol/L on day 5, 6 or 7 of stimulation or (ii) increased to a dose of 225 IU in a patient having an estradiol (E₂) level of <750 pmol/L on day 5, 6 or 7 of stimulation. Thus, the use may comprise a step of determining the serum estradiol (E₂) level of the patient on day 5, 6 or 7, and a step of administering a dose per day of menotropin (hMG) which is increased (relative to the starting dose) by up to 150 IU (for example increased by 75 IU, increased by 150 IU, or any amount in between 75 IU and 150 IU) to a patient having an estradiol (E₂) level of 750-999 pmol/L on day 5, 6 or 7 of stimulation from day 6, 7 or 8 of stimulation. Thus, the use may comprise a step of determining the serum estradiol (E₂) level of the patient on day 5, 6 or 7, and a step of administering a dose per day of menotropin (hMG) which is increased to 225 IU to a patient having an estradiol (E₂) level of <750 pmol/L on day 5, 6 or 7 of stimulation from day 6, 7 or 8 of stimulation.

From day 6, 7 or 8 of stimulation the dose per day of menotropin may be maintained (at the starting dose) in a patient having an estradiol (E₂) level of between 1000 and 3000 pmol/L on day 5, 6 or 7 of stimulation. Thus, the use may comprise a step of determining the serum estradiol (E₂) level of the patient on day 5, 6 or 7, and a step of administering a dose of, or equivalent to, 37.5 IU to 225 IU HP-hMG per day of the menotropin to a patient having an estradiol (E₂) level of between 1000 and 3000 pmol/L on day 5, 6 or 7 of stimulation from day 6, 7 or 8 of stimulation.

Thus, the use may comprise a step of determining the serum estradiol (E₂) level of the patient on day 5, 6 or 7, and a step of administering a dose of, or equivalent to, 75 IU to 225 IU HP-hMG per day of the menotropin to a patient having an estradiol (E₂) level of between 1000 and 3000 pmol/L on day 5, 6 or 7 of stimulation from day 6, 7 or 8 of stimulation.

The use may comprise a further step of triggering ovulation by administering hCG to a patient who, following stimulation, has ≥3 follicles of ≥17 mm in diameter each and estradiol (E₂) levels <10,000 pmol/L; or administering a GnRH agonist to a patient who, following stimulation, has ≥3 follicles of ≥17 mm in diameter each and estradiol (E₂) levels between 10,000 pmol/L and 15,000 pmol/L; or administering a GnRH agonist to a patient who, following stimulation, has ≥20 follicles of ≥12 mm in diameter each and/or estradiol (E₂) levels ≥15,000 pmol/mL or ≥20,000 pmol/mL. The dose of hCG to be administered to the patient may be from 5,000 IU to 10,000 IU or 150 to 500 μg recombinant hCG, for example 250 μg recombinant hCG. The use may comprise a further step of administering a GnRH antagonist starting on day 6 of stimulation.

The use may further comprise one or more or all of: retrieving (e.g. harvesting) oocyte(s); fertilizing (e.g. inseminating) the oocytes(s); allowing the fertilized oocytes to develop to the blastocyst stage; and freezing one or more (or all) blastocysts.

The menotropin and a rFSH including α2,3- and α2,6-sialylation are administered separately, for example the rFSH including α2,3- and α2,6-sialylation is for administration in the morning and the menotropin is for administration in the afternoon or evening. Alternatively, the menotropin and a rFSH including α2,3- and α2,6-sialylation are administered together.

In some aspects, the menotropin is highly purified menotropin (HP-hMG). In some aspects, the rFSH is an rFSH wherein 50 to 95% of the total sialylation is α2,3-sialylation and wherein 5-50% of the total sialylation is α2,6-sialylation. In some aspects, the rFSH is an rFSH wherein 80 to 90% of the total sialylation is α2,3-sialylation and wherein 10-20% of the total sialylation is α2,6-sialylation (e.g., follitropin delta, available from Ferring B. V.).

In some aspects, the initial daily dose of menotropin (hMG) is: 75 IU when the recombinant human follicle stimulating hormone (rFSH) including α2,3- and α2,6-sialylation daily dose is <12 μg; 150 IU when the recombinant human follicle stimulating hormone (rFSH) including α2,3- and α2,6-sialylation daily dose is 12 μg and patient body weight is <100 kg; and 225 IU when the recombinant human follicle stimulating hormone (rFSH) including α2,3- and α2,6-sialylation daily dose is 12 μg and patient body weight is ≥100 kg.

The use may comprise a further step of assessing follicular growth by ultrasonography e.g., on day 9, 10, 11, 12, 13, 14 etc. of stimulation.

In a second aspect, there is provided a product comprising a menotropin and a recombinant human follicle stimulating hormone (rFSH) including α2,3- and α2,6-sialylation; wherein the product is for use in the treatment of infertility in a patient of age≥35 (e.g., age 35 to 40, e.g. age 35-36) having a serum AMH level of ≥15 pmol/L (e.g., 15-35 pmol/L) prior to treatment/stimulation; and wherein the product is to be administered at a dose of, or equivalent to, 0.09-0.19 μg recombinant human FSH including α2,3- and α2,6-sialylation per kg body weight of the patient per day, and a dose of, or equivalent to, 37.5 IU to 225 IU HP-hMG per day of the menotropin. The dose of hMG may be a dose of, or equivalent to, 75 IU to 225 IU HP-hMG per day of the menotropin.

The use may comprise a step of determining the serum AMH level of the patient (e.g. using the automated Elecsys® AMH assay from Roche Diagnostics or similar), and a step of administering the dose of recombinant human FSH including α2,3- and α2,6-sialylation and the dose of menotropin to a patient having a serum AMH level of ≥15 pmol/L (e.g., 15-35 pmol/L).

The patient may be of age 35 to 40 years or of age>40 years. The use may comprise a step of determining the age of the patient, and a step of administering a menotropin (hMG) and a recombinant human follicle stimulating hormone (rFSH) including α2,3- and α2,6-sialylation a patient of age 35-40 years, for example 35-36 years. The use may comprise a step of determining the age of the patient, and a step of administering a menotropin (hMG) and a recombinant human follicle stimulating hormone (rFSH) including α2,3- and α2,6-sialylation to a patient of age>40 years.

The use may further comprise a step or steps of determining the serum estradiol (E₂) level of the patient during treatment/stimulation. The serum estradiol (E₂) level of the patient may be determined for the purposes of monitoring the response to stimulation and subsequently adjusting the dose per day dose of menotropin and/or for the choice of triggering medication for final follicular maturation as described in more detail below. The serum estradiol (E₂) levels may be determined via methods known in the art.

The product may be administered from day 1 of stimulation to at least day 5, 6 or 7 of stimulation. From day 6, 7 or 8 of stimulation, the product may be administered (with the dose of rFSH maintained at the (starting) day 1 dose) at a dose per day of menotropin which is maintained, increased or decreased based on the serum estradiol (E₂) level of the patient on day 5, 6 or 7 of stimulation. Thus, the use may comprise a step of determining the serum estradiol (E₂) level of the patient on day 5, 6 or 7, and a step of administering a dose per day of menotropin (human menopausal gonadotropin or hMG) which is maintained, increased or decreased relative to the starting dose to a patient from day 6, 7 or 8 of stimulation.

From day 6, 7 or 8 of stimulation, the dose per day of menotropin may be (i) decreased (relative to the starting dose) by up to 75 IU (for example decreased by 37.5 IU, decreased by 75 IU, or any amount in between 37.5 IU and 75 IU) in a patient having an estradiol (E₂) level of >3000 pmol/L on day 5, 6 or 7 of stimulation; or (ii) decreased to zero in a patient having an estradiol (E₂) level of >3000 pmol/L on day 5, 6 or 7 of stimulation. Thus, the use may comprise a step of determining the serum estradiol (E₂) level of the patient on day 5, 6 or 7, and a step of administering a dose per day of menotropin (hMG) which is (i) decreased (relative to the starting dose) by up to 75 IU (for example decreased by 37.5 IU, decreased by 75 IU, or any amount in between 37.5 IU and 75 IU) to a patient having an estradiol (E₂) level of >3000 pmol/L on day 5, 6 or 7 of stimulation from day 6, 7 or 8 of stimulation; or (ii) decreased to zero in a patient having an estradiol (E₂) level of >3000 pmol/L on day 5, 6 or 7 of stimulation from day 6, 7 or 8 of stimulation.

From day 6, 7 or 8 of stimulation, the dose per day of menotropin may be (i) increased (relative to the starting dose) by up to 150 IU (for example increased by 75 IU, increased by 150 IU, or any amount in between 75 IU and 150 IU) in a patient having an estradiol (E₂) level of 750-999 pmol/L on day 5, 6 or 7 of stimulation or (ii) increased to a dose of 225 IU in a patient having an estradiol (E₂) level of <750 pmol/L on day 5, 6 or 7 of stimulation. Thus, the use may comprise a step of determining the serum estradiol (E₂) level of the patient on day 5, 6 or 7, and a step of administering a dose per day of menotropin (hMG) which is (i) increased (relative to the starting dose) by up to 150 IU (for example increased by 75 IU, increased by 150 IU, or any amount in between 75 IU and 150 IU) to a patient having an estradiol (E₂) level of 750-999 pmol/L on day 5, 6 or 7 of stimulation from day 6, 7 or 8 of stimulation. Thus, the use may comprise a step of determining the serum estradiol (E₂) level of the patient on day 5, 6 or 7, and a step of administering a dose per day of menotropin (hMG) which is increased to 225 IU to a patient having an estradiol (E₂) level of <750 pmol/L on day 5, 6 or 7 of stimulation from day 6, 7 or 8 of stimulation.

From day 6, 7 or 8 of stimulation the dose per day of menotropin may be maintained (at the starting dose) in a patient having an estradiol (E₂) level of between 1000 and 3000 pmol/L on day 5, 6 or 7 of stimulation. Thus, the use may comprise a step of determining the serum estradiol (E₂) level of the patient on day 5, 6 or 7, and a step of administering a dose of, or equivalent to, 37.5 IU to 225 IU HP-hMG per day of the menotropin to a patient having an estradiol (E₂) level of between 1000 and 3000 pmol/L on day 5, 6 or 7 of stimulation from day 6, 7 or 8 of stimulation.

Thus, the use may comprise a step of determining the serum estradiol (E₂) level of the patient on day 5, 6 or 7, and a step of administering a dose of, or equivalent to, 75 IU to 225 IU HP-hMG per day of the menotropin to a patient having an estradiol (E₂) level of between 1000 and 3000 pmol/L on day 5, 6 or 7 of stimulation from day 6, 7 or 8 of stimulation.

The use may comprise a further step of triggering ovulation by administering hCG to a patient who, following stimulation, has ≥3 follicles of ≥17 mm in diameter each and estradiol (E₂) levels <10,000 pmol/L; or administering a GnRH agonist to a patient who, following stimulation, has ≥3 follicles of ≥17 mm in diameter each and estradiol (E₂) levels between 10,000 pmol/L and 15,000 pmol/L; or administering a GnRH agonist to a patient who, following stimulation, has ≥20 follicles of ≥12 mm in diameter each and/or estradiol (E₂) levels ≥15,000 pmol/mL or ≥20,000 pmol/mL. The dose of hCG to be administered to the patient may be from 5,000 IU to 10,000 IU or 150 to 500 μg recombinant hCG, for example 250 μg recombinant hCG. The use may comprise a further step of administering a GnRH antagonist starting on day 6 of stimulation.

The use may further comprise one or more or all of: retrieving (e.g. harvesting) oocyte(s); fertilizing (e.g. inseminating) the oocytes(s); allowing the fertilized oocytes to develop to the blastocyst stage; and freezing one or more (or all) blastocysts.

The menotropin and a rFSH including α2,3- and α2,6-sialylation are administered separately, for example the rFSH including α2,3- and α2,6-sialylation is for administration in the morning and the menotropin is for administration in the afternoon or evening. Alternatively, the menotropin and a rFSH including α2,3- and α2,6-sialylation are administered together.

In some aspects, the menotropin is highly purified menotropin (HP-hMG). In some aspects, the rFSH is an rFSH wherein 50 to 95% of the total sialylation is α2,3-sialylation and wherein 5-50% of the total sialylation is α2,6-sialylation. In some aspects, the rFSH is an rFSH wherein 80 to 90% of the total sialylation is α2,3-sialylation and wherein 10-20% of the total sialylation is α2,6-sialylation (e.g., follitropin delta, available from Ferring B. V.).

In some aspects, the initial dose of menotropin (hMG) is: 75 IU when the recombinant human follicle stimulating hormone (rFSH) including α2,3- and α2,6-sialylation dose is <12 μg; 150 IU when the recombinant human follicle stimulating hormone (rFSH) including α2,3- and α2,6-sialylation dose is 12 μg and patient body weight is <100 kg; and 225 IU when the recombinant human follicle stimulating hormone (rFSH) including α2,3- and α2,6-sialylation dose is 12 μg and patient body weight is ≥100 kg.

The use may comprise a further step of assessing follicular growth by ultrasonography e.g., on day 9, 10, 11, 12, 13, 14 etc. of stimulation.

In a further aspect, there is provided a method of treatment of infertility in a patient of age≥35 (e.g., age 35 to 40, e.g., age 35-36) and having a serum AMH level of <15 pmol/L prior to treatment, comprising; identifying a patient of age≥35 (e.g., age 35 to 40, e.g., age 35-36) and having a serum AMH level of <15 pmol/L prior to treatment; and administering to the patient human menopausal gonadotropin (hMG) and a recombinant human follicle stimulating hormone (rFSH) including α2,3- and α2,6-sialylation, wherein the rFSH is administered at a dose of, or equivalent to, 11-13 μg (e.g., 12 μg) per day of the rFSH, and the hMG is administered at a dose of, or equivalent to, 37.5 IU to 225 IU per day of highly purified hMG (HP-hMG). The dose of hMG may be a dose of, or equivalent to, 75 IU to 225 IU HP-hMG per day of the menotropin.

In a further aspect, there is provided a method of treatment of infertility in a patient of age≥35 (e.g., age 35 to 40, e.g., age 35-36) and having a serum AMH level of <15 pmol/L prior to treatment, comprising; identifying a patient of age≥35 (e.g., age 35 to 40, e.g., age 35-36) and having a serum AMH level of ≥15 pmol/L (e.g., 15-35 pmol/L) prior to treatment; and administering to the patient a product comprising human menopausal gonadotropin (hMG) and a recombinant human follicle stimulating hormone (rFSH) including α2,3- and α2,6-sialylation, wherein the rFSH is administered at a dose of, or equivalent to, 0.09-0.19 μg of the rFSH per kg body weight of the patient per day, and the hMG is administered at a dose of, or equivalent to, 37.5 IU to 225 IU per day of highly purified hMG (HP-hMG). The dose of hMG may be a dose of, or equivalent to, 75 IU to 225 IU HP-hMG per day of the menotropin.

It will be appreciated that the methods of treatment described above can be used in line with any aspects of the invention disclosed herein.

In a further aspect, there is provided a pharmaceutical composition comprising a menotropin (hMG) for use in the treatment of infertility in a patient of age≥35 (e.g., age 35 to 40, e.g., age 35-36) having a serum AMH level of <15 pmol/L prior to treatment/stimulation; wherein the pharmaceutical composition is to be administered at a dose of, or equivalent to, 37.5 IU to 225 IU HP-hMG per day of the menotropin, together with a dose of, or equivalent to, 11-13 μg (e.g., 12 μg) per day of a recombinant human FSH (rFSH) including α2,3- and α2,6-sialylation.

In a further aspect, there is provided a pharmaceutical composition comprising a recombinant human follicle stimulating hormone (rFSH) including α2,3- and α2,6-sialylation for use in the treatment of infertility in a patient of age≥35 (e.g., age 35 to 40, e.g., age 35-36) having a serum AMH level of <15 pmol/L prior to treatment/stimulation; wherein the pharmaceutical composition is to be administered at a dose of, or equivalent to, 11-13 μg (e.g., 12 μg) per day of the recombinant human FSH including α2,3- and α2,6-sialylation, together with a dose of, or equivalent to, 37.5 IU to 225 IU HP-hMG per day of a menotropin (hMG).

In a further aspect, there is provided a pharmaceutical composition comprising a menotropin (hMG) for use in the treatment of infertility in a patient of age≥35 (e.g., age 35 to 40, e.g., age 35-36) having a serum AMH level of ≥15 pmol/L (e.g., 15-35 pmol/L) prior to treatment/stimulation; wherein the pharmaceutical composition is to be administered at a dose of, or equivalent to, 37.5 IU to 225 IU HP-hMG per day of the menotropin, together with a dose of, or equivalent to, 0.09-0.19 μg per kg body weight of the patient per day of a recombinant human FSH including α2,3- and α2,6-sialylation.

In a further aspect, there is provided a pharmaceutical composition comprising a recombinant human follicle stimulating hormone (rFSH) including α2,3- and α2,6-sialylation for use in the treatment of infertility in a patient of age≥35 (e.g., age 35 to 40, e.g., age 35-36) having a serum AMH level of ≥15 pmol/L (e.g., 15-35 pmol/L) prior to treatment/stimulation; wherein the pharmaceutical composition is to be administered at a dose of, or equivalent to, 0.09-0.19 μg per kg body weight of the patient per day of the recombinant human FSH including α2,3- and α2,6-sialylation, together with a dose of, or equivalent to, 37.5 IU to 225 IU HP-hMG per day of the menotropin (hMG).

It will be appreciated that the pharmaceutical compositions described above represent components of the product according to other aspects of the invention and can be used in line with any aspect of the invention disclosed herein.

### Menotropin and Recombinant FSH and rFSH Compositions

The methods and products described herein use recombinant human follicle stimulating hormone (rFSH) including α2,3- and α2,6-sialylation. FSH comprises a 92 amino acid alpha sub-unit, also common to the other glycoprotein hormones LH and chorionic gonadotropin (CG), and a 111 amino acid beta sub-unit unique to FSH that confers the biological specificity of the hormone (Pierce and Parsons, 1981). Each sub-unit is post translationally modified by the addition of complex carbohydrate residues. Both subunits carry 2 sites for N-linked glycan attachment, the alpha sub-unit at amino acids 52 and 78 and the beta sub-unit at amino acid residues 7 and 24 (Rathnam and Saxena, 1975, Saxena and Rathnam, 1976). FSH is thus glycosylated to about 30% by mass (Dias and Van Roey. 2001. Fox et al. 2001).

The glycosylation of rFSH products reflects the range of glycosyl-transferases present in the host cell line. Commercially available rFSH products derived from engineered Chinese hamster ovary cells (CHO cells) have a more limited range of glycan modifications than those found on the natural products. Examples of the reduced glycan heterogeneity found in CHO cell-derived rFSH include a lack of bisecting glucosamine and a reduced content of core fucosylation and acetyl lactosamine extensions (Hard et al., 1990). In addition, CHO cells are only able to add sialic acid at the α2,3 linkage (Kagawa et al, 1988, Takeuchi et al, 1988, Svensson et al., 1990); thus, CHO cell-derived rFSH only includes α2,3-linked sialic acid and does not include α2,6-linked sialic acid. Thus, CHO cell-derived rFSH is different from naturally produced FSH (e.g., human pituitary/serum/urinary FSH) which contains glycans with a mixture of α2,3 and α2,6-linked sialic acid (i.e., α2,3- and α2,6-sialylation), with a predominance of the former.

The present applicants have developed a human cell line-derived rFSH which is the subject of International Patent Application No. PCT/GB2009/000978, published as WO2009/127826A, and also approved by the European Commission as REVOKELLE® (follitropin delta) (also known as FE 999049). This recombinant FSH, with a mixture of both α2,3 and α2,6-linked sialic acid (i.e., α2,3- and α2,6-sialylation), was made by engineering a human cell line to express both rFSH and α2,3-sialyltransferase. The amino acid sequence of the human cell line-derived recombinant FSH which is the subject of International Patent Application No. PCT/GB2009/000978, published as WO2009/127826A (e.g., follitropin delta/FE 999049), is the native human FSH sequence, but the product has a different glycosylation pattern. The expressed product is highly acidic and carries a mix of both α2,3- and α2,6-linked sialic acids (i.e., α2,3- and α2,6-sialylation); the latter provided by the endogenous sialyl transferase activity. It was found that the type of sialic acid linkage, α2,3-or α2,6-, can have a dramatic influence on biological clearance of FSH. Thus REVOKELLE® (follitropin delta/FE 999049) may be more biologically appropriate compared to CHO cell-derived recombinant products that have only α2,3 linked sialic acid (Kagawa et al, 1988, Takeuchi et al, 1988, Svensson et al., 1990) and have decreased sialic acid content (Ulloa-Aguirre et al. 1995., Andersen et al. 2004).

Thus, the recombinant FSH including α2,3- and α2,6-sialylation used in accordance with the methods and products described herein may be produced or expressed in a human cell line, such as a PER.C6® cell line. The recombinant FSH may be produced or expressed in a PER.C6® cell line, a PER.C6® derived cell line or a modified PER.C6® cell line. Recombinant FSH which is produced or expressed in a PER.C6® cell line will include some α2,6-linked sialic acids (α2,6 sialylation) provided by endogenous sialyl transferase activity (of the cell line) and will include some α2,3-linked sialic acids (α2,3 sialylation) provided by endogenous sialyl transferase activity. The cell line may be modified using α2,3-sialyltransferase. The cell line may be modified using α2,6-sialyltransferase. Alternatively or additionally, the recombinant FSH may include α2,6-linked sialic acids (α2,6 sialylation) provided by endogenous sialyl transferase activity (of the cell line). Herein, the term “human-derived recombinant FSH” means recombinant FSH which is produced or expressed in a human cell line (e.g., recombinant FSH made by engineering a human cell line).

The recombinant FSH used in the methods and products described herein may include α2,3- and α2,6-sialylation. The recombinant FSH for use according to the invention may have 1% to 99% of the total sialylation being α2,3-sialylation. The recombinant FSH for use according to the invention may have 1% to 99% of the total sialylation being α2,6-sialylation. The recombinant FSH may have 1% to 50% of the total sialylation as α2, 6-sialyation, and 50% to 99% of the total sialylation as 2,3-sialyation. For example, 80% to 95%, for example 80% to 90%, for example 82% to 89%, for example 85% to 89% of the total sialylation may be α2,3-sialylation. For example, 5% to 20%, for example 10% to 20%, for example 11% to 18%, for example 11% to 15%, of the total sialylation may be α2,6-sialylation. In an example, the recombinant FSH has 5% to 20% of the total sialylation as α2, 6-sialyation, and 80% to 95% of the total sialylation as 2,3-sialyation. In another example, the recombinant FSH has 50% to 80% of the total sialylation as α2, 6-sialyation, and 20% to 50% of the total sialylation as 2,3-sialyation.

Herein, by “sialylation”, it is meant the amount of sialic residues present on the recombinant FSH carbohydrate structures. Consistent with usage in the art, α2,3-sialylation means sialylation at the 2,3 position and α2,6 sialylation means sialylation at the 2,6 position. Thus “% of the total sialylation may be a 2,3 sialylation” refers to the % of the total number of sialic acid residues present in the FSH (or hCG) which are sialylated in the 2,3 position. The term “% of the total sialylation being α2,6-sialylation” refers to the % of the total number of sialic acid residues present in the FSH (or hCG) which are sialylated in the 2,6 position.

In all aspects, the rFSH may be present as a single isoform or as a mixture of isoforms.

The recombinant FSH and hMG can be formulated into well-known compositions for any route of drug administration, e.g., oral, rectal, parenteral, transdermal (e.g., patch technology), intravenous, intramuscular, subcutaneous (e.g., for subcutaneous injection), intracisternal, intravaginal, intraperitoneal, local (powders, ointments or drops) or as a buccal or nasal spray. A typical composition comprises a pharmaceutically acceptable carrier, such as aqueous solution, nontoxic excipients, including salts and preservatives, buffers and the like, as described in Remington's Pharmaceutical Sciences fifteenth edition (Matt Publishing Company, 1975), at pages 1405 to 1412 and 1461-87, and the national formulary XIV fourteenth edition (American Pharmaceutical Association, 1975), among others. For example, the recombinant FSH, composition or pharmaceutical composition can be formulated for injection, such as for subcutaneous injection.

Examples of suitable aqueous and non-aqueous pharmaceutical carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate.

The composition may also comprise additives such as but not limited to preservatives, wetting agents, emulsifying agents, surfactants and dispersing agents. Antibacterial and antifungal agents can be included to prevent growth of microbes and include, for example, m-cresol, benzyl alcohol, paraben, chlorobutanol, phenol, sorbic acid, and the like. If a preservative is included, benzyl alcohol, phenol and/or m-cresol are preferred; however, the preservative is by no means limited to these examples. Furthermore, it may be desirable to include isotonic agents such as sugars, sodium chloride, amino acids and the like.

For example, the composition or medicament may comprise recombinant FSH and one or more of polysorbate 20, L-methionine, phenol, and arginine hydrochloride. Such a composition may be formulated for injection, such as for subcutaneous injection. For example, the rFSH composition or medicament may be the REKOVELLE® formulation (follitropin delta with excipients phenol, polysorbate 20, L-methionine, sodium sulphate decahydrate, disodium phosphate dodecahydrate, phosphoric acid [concentrated, for pH-adjustment], sodium hydroxide [for pH-adjustment], and water for injection).

Injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use. Injectable formulations can be supplied in any suitable container, e.g., vial, pre-filled syringe, injection cartridges, and the like.

The recombinant FSH, composition, or medicament may be formulated for single use or for multiple use (multiple dose). If the recombinant FSH, composition, or medicament is formulated for multiple use, typically one or more preservatives is included. If a preservative is included, benzyl alcohol, phenol or m-cresol, are preferred; however, the preservative is by no means limited to these examples. The single use or multiple use formulated composition or medicament may further comprise an amino acid or combination of amino acids. Typically, the amino acid is arginine, for example added as arginine or more typically arginine hydrochloride.

The recombinant FSH, composition, or medicament may be included in a container such as a vial, prefilled cartridge (e.g., for single administration or multiple use) or an injection device such as a “pen” for e.g., administration of multiple doses.

The recombinant FSH, composition or pharmaceutical composition may be a formulation (e.g., injectable formulation) including rFSH.

The recombinant FSH, composition or medicament can be supplied in any appropriate package. For example, a composition or medicament can include a number of containers (e.g., pre-filled syringes or vials) containing FSH. The syringes or vials may be packaged in a blister package or other means to maintain sterility. Any composition or medicament can optionally include instructions for using the FSH formulation.

The pH and exact concentration of the various components of the pharmaceutical composition are adjusted in accordance with routine practice in this field. See GOODMAN and GILMAN's THE PHARMACOLOGICAL BASIS FOR THERAPEUTICES, 7^{th} ed. In a typical embodiment, the recombinant FSH, composition or medicament are supplied as compositions for parenteral administration. General methods for the preparation of the parenteral formulations are known in the art and are described in REMINGTON; THE SCIENCE AND PRACTICE OF PHARMACY, supra, at pages 780-820. The parenteral compositions can be supplied in liquid formulation or as a solid which will be mixed with a sterile injectable medium just prior to administration. The parenteral compositions may be supplied in dosage unit form for ease of administration and uniformity of dosage.

In a further aspect there is provided the use of recombinant follicle stimulating hormone (FSH) in the manufacture of a medicament for the uses as described herein.

Further aspects are illustrated in the following examples, which are not limiting in any respect.

The menotropin, e.g., human menopausal gonadotropin (hMG), used in the methods and products described herein may be highly purified menotropin. The term “highly purified menotropin” as used herein includes HP-hMG available under the trademark MENOPUR® from Ferring B. V., that contains both follicle stimulating hormone (FSH) and human chorionic gonadotropin (hCG)-driven luteinizing hormone (LH)-activity. In some examples, the hMG used in the methods and products described herein is a HP-hMG which includes both follicle stimulating hormone (FSH) and human chorionic gonadotropin (hCG)-driven luteinizing hormone (LH)-activity (e.g. MENOPUR®). In some examples, the hMG used in the methods and products described herein is the MENOPUR® HP-hMG product, which is typically provided as a lyophilized powder or pellet to be mixed with sterile diluent (e.g., 0.9% Sodium Chloride for Injection) prior to use. MENOPUR® is a powder and solvent solution for injection. The ingredients in MENOPUR® in addition to hMG are lactose, polysorbate 20, hydrochloric acid, sodium chloride, sodium hydroxide and water for injections.

### EXAMPLES

The following examples use REKOVELLE® (follitropin delta), which is a recombinant FSH expressed in a PER.C6® cell line engineered by the methods disclosed in WO2013/020996 and WO2009/127826A, and MENOPUR®, which is highly purified menotropin (HP-hMG) that contains both follicle stimulating hormone (FSH) and human chorionic gonadotropin (hCG)-driven luteinizing hormone (LH)-activity.

The Marketing Authorisation holder for REKOVELLE® and MENOPUR® is Ferring Pharmaceuticals A/S of Kay Fiskers Plads 11, 2300 Copenhagen S, Denmark, and REKOVELLE® is available in the UK from Ferring Pharmaceuticals of Drayton Hall, Church Road, West Drayton, UB7 7PS, UK.

The active substance in REKOVELLE® is follitropin delta (FE999049). REKOVELLE® is highly sialylated and includes α2,3- and α2,6-sialylation, with about 85% to 90% of the total sialylation being α2,3-sialylation and about 10% to 15% of the total sialylation being α2,6-sialylation.

REKOVELLE® is a clear and colourless solution for injection (injection). One millilitre of solution contains 33.3 micrograms of follitropin delta in each millilitre of solution. The other ingredients are phenol, polysorbate 20, L-methionine, sodium sulphate decahydrate, disodium phosphate dodecahydrate, concentrated phosphoric acid, sodium hydroxide and water for injections.

The active substance in MENOPUR® is human menopausal gonadotrophin (HMG).

MENOPUR® is a powder and solvent solution for injection. The other ingredients are lactose, polysorbate 20, hydrochloric acid, sodium chloride, sodium hydroxide and water for injections. MENOPUR® (e.g. MENOPUR® 75 IU injection, Menopur® 1200 IU Powder and solvent for solution for injection) is available in the UK from Ferring Pharmaceuticals Limited of Drayton Hall, Church Road, West Drayton, UB7 7PS, UK.

### Example 1: Menopur and Rekovelle Combined Study (MARCS): An Exploratory Study Evaluating the Effect of Individualised Dosages of Follitropin Delta and Highly Purified Human Menopausal Gonadotropin (HP-hMG) for Controlled Ovarian Stimulation

### Introduction

The MARCS study aimed to evaluate the mean number of good-quality blastocysts obtained at day 5 and on day 6 as well as the proportion of women with ovarian hyperstimulation syndrome (OHSS) after treatment with an individualised dosing regimen of a follitropin delta and highly purified human menopausal gonadotrophin (HP-hMG) compared with treatment with personalised doses of follitropin delta used as monotherapy in the Evidence-based Stimulation Trial with Human Recombinant Follicle-Stimulating Hormone in Europe and Rest of World 1 (ESTHER-1) trial (Nyboe Andersen A, Nelson S M, Fauser B C, García-Velasco J A, Klein B M, Arce J C; ESTHER-1 study group. Individualized versus conventional ovarian stimulation for in vitro fertilization: a multicenter, randomized, controlled, assessor-blinded, phase 3 noninferiority trial. Fertil Steril. 2017 February;107 (2): 387-396).

### Methods

This was a multicenter, open label, single-cohort with external (synthetic) control study assessing the efficacy and safety of personalized dosage of follitropin delta in combination with HP-hMG (“mixed protocol”). Patients were enrolled at four research sites in Canada. The study protocol was approved by local regulatory authorities and independent ethics committees. The study was performed in accordance with the principles of the Declaration of Helsinki, the International Conference on Harmonization Guidelines for Good Clinical Practice, and local regulatory requirements. All participants provided written, informed consent prior to undergoing any study related procedures.

### Study Participants

Women enrolled in the study were between 18-40 years of age, had a confirmed diagnosis of infertility (including stage I/II endometriosis or tubal factor) and were undergoing their first IVF/intracytoplasmic sperm injection (ICSI) cycle. Additional inclusion criteria were regular menstrual cycles of 24-35 days, presence of both ovaries, use of ejaculated sperm (fresh or frozen) for fertilization, and early follicular phase FSH serum concentration <10 IU/L. The main exclusion criteria were endometriosis stage III/IV, high risk for OHSS (AMH >35 pmol/L), history of recurrent miscarriages, defined as ≥3 consecutive losses, use of hormonal treatment (except for thyroid medication) during the last menstrual cycle before enrolment in the study.

### Study Procedures

All patients participating in the study were treated with a fixed personalized daily subcutaneous dose of follitropin delta (REKOVELLE®, Ferring Pharmaceuticals A/S), determined by an established algorithm based on body weight and serum AMH level (Table 1) which is the subject of International Patent Application No. PCT/EP2012/065507, published as WO2013/020996. Serum AMH measurements were performed at a central laboratory (CReATe Fertility Centre), using the automated Elecsys AMH immunoassay (Roche), and were determined within the last 12 months before the start of ovarian stimulation.

TABLE 1
Calculation of daily dose of follitropin delta based on AMH concentration

| | Daily dose of follitropin delta fixed throughout | |
|---|---|---|
| AMH concentration (pmol/L) | stimulation | |
| <15 | 12 | μg |
| 15-16 | 0.19 | μg/kg |
| 17 | 0.18 | μg/kg |
| 18 | 0.17 | μg/kg |
| 19-20 | 0.16 | μg/kg |
| 21-22 | 0.15 | μg/kg |
| 23-24 | 0.14 | μg/kg |
| 25-27 | 0.13 | μg/kg |
| 28-32 | 0.12 | μg/kg |
| 33-39 | 0.11 | μg/kg |
| ≥40 | 0.10 | μg/kg |

The dose is rounded off to the nearest 0.33 micrograms to match the dosing scale on the injection pen.

HP-hMG (MENOPUR®, Ferring Pharmaceuticals A/S) was co-administered with follitropin delta to all patients at personalized initial doses according to the patient's weight and the calculated dose of follitropin delta.

The initial daily dose of HP-hMG was:
75 IU when the follitropin delta dose was <12 μg;
150 IU when the follitropin delta daily dose was 12 μg and body weight was <100 kg;
225 IU when the follitropin delta daily dose was 12 μg and body weight was ≥100 kg.

Gonadotropin therapy was initiated on day 2 of the menstrual cycle. A gonadotropin-releasing hormone (GnRH) antagonist was initiated on day 6 of stimulation and continued throughout the remainder of stimulation. The response to stimulation was monitored via serum estradiol (E₂) levels and vaginal sonography.

As this was an exploratory study, adjustments in the medication regime were expected. Any adjustment in stimulation was confined to HP-hMG and was allowed only starting from day 6 of stimulation. The HP-hMG dose could be adjusted up or down according to the E₂ level on day 6 of stimulation regardless of follicular development, with 225 IU as the maximum daily dose. The HP-hMG dose adjustments were done according to the schedule described in Table 2.

TABLE 2
HP-hMG day 6 dose adjustment chart

| Estradiol level at day 6 of stimulation | | |
|---|---|---|
| pmol/L | pg/mL | HP-hMG Dosage |
| 1000 and 3000 pmol/L | 272.38 and 817.14 pg/mL | Initial dose is continued |
| >3000 pmol/L | >817.14 pg/mL | Decrease dosage by 75 IU* |
| <1000 pmol/L | <272.38 pg/mL | Increase dosage by 75 IU |
| If E₂ between 750 and 999 | If E₂ between 204.28 and | Increase dosage to 225 IU |
| pmol/L | 272.10 pg/mL | |
| If E₂ < 750 pmol/L | If E₂ < 204.28 pg/mL | |

*If the patient was on 75 IU, dose was decreased to 37.5 IU or withdrawn completely

Triggering of final follicular maturation was performed when ≥3 follicles reached 17 mm in diameter. The choice of the triggering medication was based on serum E₂ levels before trigger: hCG 5,000-10,000 IU if E₂<10,000 pmol/L; and GnRH agonist 0.2 mg (triptorelin acetate, Decapeptyl; Ferring Pharmaceuticals) if E₂≥10,000 pmol/L. In case of excessive response, defined as serum levels of E₂≥15,000 pmol/L on the day of trigger or ≥20 follicles of ≥12 mm, a freeze-all strategy was employed. In the case of poor follicular development, defined as <3 follicles of >17 mm reached by day 20, the cycle was cancelled.

Oocyte retrieval took place 36±2 hours after triggering of final follicular maturation. Oocytes were inseminated by IVF or ICSI using ejaculated sperm from a partner or donor. All embryos were cultured up to day 5 or 6. All fresh embryo transfers were performed on day 5 of culture, and all were single-embryo transfers.

Surplus blastocysts were cryopreserved for future use when the grade was 3BB or higher according to the Gardner classification. Daily intramuscular progesterone was prescribed for luteal phase support. Adverse events were recorded from the time of signing the informed consent until the end of the study, defined as day-6 blastocyst formation. If any adverse event was reported during the study visits, follow-up calls were done by the research team until the resolution of the adverse event.

### Study Outcomes

The primary endpoint of the study was the number of good-quality (>3BB) blastocysts (usable blastocysts) at day 5 and on day 6. The secondary objectives were the ovarian response, embryology and safety of the regime. Ovarian response and embryology included duration of stimulation, total doses of follitropin delta, percentage of patients triggered with GnRH agonist, the mean number of mature oocytes obtained, the mean number of embryos on day 3, and the percentage of day 3 embryos that evolved into usable blastocysts on day 5 or day 6 (attrition rate). Safety was assessed by evaluating the incidence and severity of early and late onset moderate to severe OHSS, classified according to Golan's system (see Table 3).

TABLE 3
OHSS classification
Ovarian hyperstimulation syndrome classification (17)

| | |
|---|---|
| Mild | Grade 1: abdominal distension and discomfort |
| | Grade 2: grade 1 + nausea, vomiting and/or diarrhea, enlarged ovaries (5-12 |
| | cm) |
| Moderate | Grade 3: grade 2 + ultrasound evidence of ascites |
| Severe | Grade 4: grade 3 + clinical evidence of ascites and/or hydrothorax and |
| | breathing difficulties |
| | Grade 5: grade 4 + hemoconcentration, increased blood viscosity, coagulation |
| | abnormality and diminished renal perfusion |

### Results

This study employed a single-cohort, open-label design with an external (synthetic) control as a comparator. The patients enrolled in the ESTHER-1 trial who were treated with personalized doses of follitropin delta were used as the comparators. Table 4 shows the primary and secondary endpoints by cohort of MARCS compared to ESTHER-1 and Table 5 shows the primary and secondary end points of the MARCS cohort distributed by age group.

TABLE 4
Primary and secondary end points by cohort

| | Cohort | | | | |
|---|---|---|---|---|---|
| | MARCS | | ESTHER-1 | | P value |
| Characteristic | (n = 110)^{a} | | (n = 665)^{b} | | MARCS vs. ESTHER-1^{b} |
| Primary end points | | | | | |
| Good-quality usable | 4.9 ± 3.9^{d} | | 2.0 ± 2.2 | | <.001 |
| blastocysts at day 5-6^{c} | | | | | |
| Sensitivity analysis | | | | | |
| Good-quality usable | 5.39 ± 2.0^{d} | | 2.0 ± 2.2 | | <.001 |
| blastocysts at day 5-6 | | | | | |
| Secondary end points | | | | | |
| Duration of stimulation (d) | 11.33 ± 1.29^{e} | | 8.9 ± 1.9 | | <.001 |
| Follitropin delta total dose (μg) | 123.72 ± 21.75^{e} | | 90.0 ± 25.3 | | <.001 |
| Women with investigator- | 67 | (39.1%) | 221 | (33.2%)^{f} | <.001 |
| requested gonadotropin dose | | | | | |
| adjustments | | | | | |
| Poor response leading to cycle | 3 | (2.7%) | 25 | (3.8%) | .289 |
| cancellation | | | | | |
| Triggering with GnRH agonist | 46 | (43%)^{e} | 10 | (1.5%) | <.001 |
| Oocytes retrieved CCOO | 14.55 ± 7.58^{e} | | 10.0 ± 5.6 | | <.001 |
| Mature oocytes retrieved (MII) | 11.28 ± 5.76^{e} | | 7.4 ± 4.3^{g} | | <.001 |
| Embryos on day 3 | 8.30 ± 5.05^{d} | | 5.4 ± 3.7 | | <.001 |
| Attrition rates between day 3 | 59.03%^{d} | | 37.03% | | <.001 |
| and blastocyst | | | | | |

Note:
Values are mean ± standard deviation or number and percentage unless otherwise indicated. CCOO = cumulus oophorous; ESTHER-1 = Evidence-based Stimulation Trial with Human rFSH in Europe and Rest of World 1; GnRH = gonadotropin-releasing hormone; MARCS = Menopur and Rekovelle Combined Study; MII = metaphase II.
^{a}Where N is the number of participants taken into account for each variable differs in the cases noted by d and e).
^{b}ESTHER-1 (P) group with personalized follitropin delta dose.
^{c}Observed/unadjusted estimates.
^{d}N = 106 patients. Four patients did not obtain usable embryo on day 5 or 6
^{e}N = 107 patients. Three patients obtained a poor response leading to cycle cancellation.
^{f}Although an adjustment was requested by an investigator, no adjustment was done in the ESTHER-1 (P) group as per protocol.
^{g}Data from clinical trial report.

TABLE 5
Primary and secondary end points distributed by age group

| | Age group (y) | | | | | | | | |
|---|---|---|---|---|---|---|---|---|---|
| Characteristic | 18.0-34.9 | | 35.0-36.9 | | 37.0-40.0 | | Total | | P value |
| N (%) | 55 | (50%) | 24 | (22%) | 31 | (28%) | 110 | (100%)^{a} | |
| No. of good-quality | | | | | | | | | |
| blastocysts | | | | | | | | | |
| Day 5-6 (unadjusted) | 6.08 ± 4.23 | | 4.22 ± 3.49 | | 3.45 ± 2.84 | | 4.91 ± 3.87^{c} | | .006 |
| Day 5-6 (adjusted)^{b} | 5.36 ± 0.49 | | 4.03 ± 0.72 | | 4.24 ± 0.62 | | NA | | .244 |
| Secondary end points | | | | | | | | | |
| Follitropin delta dose/d | 10.48 ± 2.06 | | 11.35 ± 1.69 | | 11.38 ± 1.7 | | 10.92 ± 1.92^{d} | | .054 |
| HP-hMG dose/d | 120 ± 39.79 | | 146.88 ± 41.25 | | 147.58 ± 36.14 | | 133.64 ± 41.12^{d} | | .002 |
| Days of stimulation | 11.23 ± 1.12 | | 11.57 ± 1.50 | | 11.32 ± 1.42 | | 11.33 ± 1.29^{d} | | .581 |
| No. of follicles | | | | | | | | | |
| <15 mm^{a} | 11.58 ± 9.64 | | 6.87 ± 7.10 | | 4.33 ± 4.81 | | 8.51 ± 8.56^{d} | | <.001 |
| ≥15 mm^{a} | 10.72 ± 5.38 | | 7.57 ± 3.27 | | 7.77 ± 3.69 | | 9.20 ± 4.76^{d} | | .004 |
| Endometrium thickness | 11.18 ± 2.11 | | 11.11 ± 2.56 | | 10.54 ± 2.04 | | 11.05 ± 2.24^{d} | | .296 |
| (mm) | | | | | | | | | |
| E₂ at day 6 of stimulation | 1,760 ± 1,134 | | 1392 ± 907 | | 1,563 ± 1,060 | | 1,624 ± 1,068^{d} | | .348 |
| pg/mL | 479 ± 309 | | 379 ± 247 | | 426 ± 289 | | 442 ± 291 | | |
| Final E₂ | 11,090 ± 5,278 | | 9,742 ± 5,970 | | 8,952 ± 4,558 | | 10,181 ± 5,276^{d} | | .183 |
| pg/mL | 3,021 ± 1,438 | | 2,654 ± 1,626 | | 2,438 ± 1,242 | | 2,773 ± 1,437 | | |
| HP-hMG dose adjustment | | | | | | | | | |
| No change | 33 | (60%) | 17 | (70.83%) | 17 | (54.84%) | 67 | (60.9%) | .410 |
| 225 IU | 13 | (23.64%) | 7 | (29.17%) | 11 | (35.48%) | 31 | (28.2%) | |
| 150 IU | 3 | (5.45%) | 0 | (0) | 0 | (0) | 3 | (2.7%) | |
| 75 IU | 2 | (3.64%) | 0 | (0) | 2 | (6.45%) | 4 | (3.6%) | |
| 37.5 IU | 4 | (7.27%) | 0 | (0) | 1 | (3.23%) | 5 | (4.5%) | |
| Withdrawal of HP-hMG | 0 | (0) | 0 | (0) | 0 | (0) | 0 | (0) | |
| Trigger medication | | | | | | | | | |
| Decapeptyl 0.2 mg | 29 | (54.72%) | 6 | (26.09%) | 11 | (35.48%) | 46 | (43%) | .024 |
| hCG 5,000 IU | 15 | (28.30%) | 5 | (21.74%) | 8 | (25.81%) | 28 | (26.2%) | |
| hCG 10,000 IU | 9 | (16.98%) | 12 | (52.17%) | 12 | (38.71%) | 33 | (30.8%) | |
| No. of CCOO | 17.79 ± 8.23 | | 12.39 ± 5.11 | | 10.61 ± 5.32 | | 14.55 ± 7.58^{d} | | <.001 |
| No. of oocytes (MII) | 13.13 ± 6.24 | | 10.17 ± 5.02 | | 8.94 ± 4.34 | | 11.28 ± 5.76^{d} | | .003 |
| No. of embryos at day 3 | 9.66 ± 5.48 | | 7.96 ± 4.51 | | 6.23 ± 3.92 | | 8.30 ± 5.05^{c} | | .009 |
| Attrition rates between | 62.94% | | 53.02% | | 55.37% | | 59.16%^{c} | | .411 |
| day 3 and blastocyst | | | | | | | | | |
| Safety outcomes | | | | | | | | | |
| OHSS^{b} | | | | | | | | | |
| None | 48 | (88.89%) | 21 | (91.30%) | 29 | (93.55%) | 98 | (90.7%) | .771 |
| Mild grade 1 | 6 | (11.11%) | 2 | (8.70%) | 2 | (6.45%) | 10 | (9.3%) | |
| Mild grade 2 | 0 | (0) | 0 | (0) | 0 | (0) | 0 | (0) | |
| Transfer type^{b} | | | | | | | | | |
| No transfer (no | 0 | (0) | 2 | (8.70%) | 2 | (6.45%) | 4 | (3.7%) | .164 |
| blastocysts available) | | | | | | | | | |
| Fresh embryo transfer | 17 | (32.08%) | 10 | (43.48%) | 8 | (25.81%) | 34 | (32.7%) | |
| Freeze all | 36 | (67.92%) | 11 | (47.83%) | 21 | (67.74%) | 68 | (63.6%) | |

Note:
Values are mean ± standard deviation or number and percentage unless otherwise indicated. CCOO = cumulus oophorous; E2 = estradiol; HP-hMG = highly purified human menopausal gonadotropin; MII = metaphase II; OHSS = ovarian hyperstimulation syndrome.
^{a}N = The number of participants taken into account for each variable differs in the cases marked with c and d.
^{b}Least square mean ± standard error of the mean, adjusted for antral follicle count, and antimüllerian hormone level.
^{c}N = 106 patients. Four patients did not obtain usable embryo on day 5 or 6
^{d}N = 107 patients. Three patients obtained a poor response leading to cycle cancellation.

Table 4 and 5 show that the number of good-quality, usable blastocysts obtained by day 5 or 6 of embryo culture can be statistically significantly increased by using the mixed protocol described herein without compromising safety, compared with cycles where follitropin delta was used as monotherapy.

The subgroup of 35- to 36-year-old and 37- to 40-year-old patients are the ones that appear to benefit the most from the application of the combination regimen. For example, the mean (adjusted) number of good-quality blastocysts on day 5-6 after fertilisation was 4.03±0.72 and 4.24±0.62 for the 35.0-36.9 and 37.0-40.0 age group respectively compared to 2.0±2.2 (for all age groups) where follitropin delta was used as monotherapy. These results suggest that women aged older than 35 years would experience the greatest benefit in terms of oocytes retrieved from the combined dosing protocol described herein and assessed in this study.

The incidence of any grade of OHSS was 9.3% (7.4% for the older age groups, 35.0-36.9 and 37.0-40.0) compared to 2.6% where follitropin delta was used as monotherapy. No cases of moderate or severe OHSS were observed in the study compared with cases in 1.4% of patients where follitropin delta was used as monotherapy. Thus, the dosing regimen described herein achieves the desired goal.

The percentage of patients who did not require dose adjustment of menotropin (HP-hMG) was 70.83% and 54.84% for the 35.0-36.9 and 37.0-40.0 age group respectively. As the study was exploratory, adjustments in the medication regime were expected, therefore it is remarkable that no change in HP-hMG dose was required for this percentage of these patients.

### Conclusion

Optimizing ovarian response in IVF by employing a mixed protocol of individualized dosing of both follitropin delta and HP-hMG results in a statistically significant increase in the number of good-quality usable blastocysts on day 5 or 6 with an increased risk of only mild OHSS, which has not required medical intervention or hospitalization. The subgroup of 35- to 36-year-old and 37- to 40-year-old patients are the ones that appear to benefit the most from the application of this combination regimen.

1. A product comprising a menotropin (human menopausal gonadotropin or hMG) and a recombinant human follicle stimulating hormone (rFSH) including α2,3- and α2,6-sialylation; wherein the product is for use in the treatment of infertility in a patient of age≥35 (e.g., age 35 to 40, e.g., age 35-36) having a serum AMH level of <15 pmol/L prior to treatment/stimulation; wherein the product is to be administered at a dose of, or equivalent to, 11-13 μg (e.g., 12 μg) per day of the recombinant human FSH including α2,3- and α2,6-sialylation, and a dose of, or equivalent to, 37.5 IU to 225 IU HP-hMG per day of the menotropin.

2. A product for use according to claim 1 wherein the use comprises a step of determining the serum AMH level of the patient, and a step of administering the dose of recombinant human FSH including α2,3- and α2,6-sialylation and the dose of menotropin to a patient having a serum AMH level of <15 pmol/L.

3. A product comprising a menotropin and a recombinant human follicle stimulating hormone (rFSH) including α2,3- and α2,6-sialylation; wherein the product is for use in the treatment of infertility in a patient of age≥35 (e.g., age 35 to 40, e.g. age 35-36) having a serum AMH level of ≥15 pmol/L prior to treatment/stimulation; and wherein the product is to be administered at a dose of, or equivalent to, 0.09-0.19 μg recombinant human FSH including α2,3- and α2,6-sialylation per kg body weight of the patient per day, and a dose of, or equivalent to, 37.5 IU to 225 IU HP-hMG (e.g. 75 IU) per day of the menotropin.

4. A product for use according to claim 3 wherein the use comprises a step of determining the serum AMH level of the patient, and a step of administering the dose of recombinant human FSH including α2,3- and α2,6-sialylation and the dose of menotropin to a patient having a serum AMH level of ≥15 pmol/L.

5. A product for use according to any preceding claim wherein the use comprises a further step of determining the serum estradiol (E₂) level of the patient during treatment/stimulation.

6. A product for use according to any preceding claim wherein the product is to be administered from day 1 of stimulation to at least day 5, 6 or 7 of stimulation.

7. A product for use according to claim 6 wherein from day 6, 7 or 8 of stimulation, the product is to be administered at a dose per day of menotropin which is maintained, increased or decreased based on the serum estradiol (E₂) level of the patient on day 5, 6 or 7 of stimulation.

8. A product for use according to claim 7 wherein from day 6, 7 or 8 of stimulation, the dose per day of menotropin is (i) decreased (relative to the starting dose) by up to 75 IU (for example decreased by 37.5 IU, decreased by 75 IU, or any amount in between 37.5 IU and 75 IU) in a patient having an estradiol (E₂) level of >3000 pmol/L on day 5, 6 or 7 of stimulation; or (ii) decreased to zero in a patient having an estradiol (E₂) level of >3000 pmol/L on day 5, 6 or 7 of stimulation.

9. A product for use according to claim 7 wherein from day 6, 7 or 8 of stimulation, the dose per day of menotropin is (i) increased (relative to the starting dose) by up to 150 IU (for example increased by 75 IU, increased by 150 IU, or any amount in between 75 IU and 150 IU) in a patient having an estradiol (E₂) level of 750-999 pmol/L on day 5, 6 or 7 of stimulation or (ii) increased (relative to the starting dose) to a dose of 225 IU in a patient having an estradiol (E₂) level of <750 pmol/L on day 5, 6 or 7 of stimulation.

10. A product for use according to claim 7 wherein from day 6, 7 or 8 of stimulation the dose per day of menotropin is maintained in a patient having an estradiol (E₂) level of between 1000 and 3000 pmol/L on day 5, 6 or 7 of stimulation.

11. A product for use according to any preceding claim wherein the use comprises a further step of triggering ovulation by administering hCG to a patient who, following stimulation, has ≥3 follicles of ≥17 mm in diameter each and estradiol (E₂) levels <10,000 pmol/L; or administering a GnRH agonist to a patient who, following stimulation, has ≥3 follicles of ≥17 mm in diameter each and estradiol (E₂) levels between 10,000 pmol/L and 15,000 pmol/L; or administering a GnRH agonist to a patient who, following stimulation, has ≥20 follicles of ≥12 mm in diameter each and/or estradiol (E₂) levels ≥15,000 pmol/mL or ≥20,000 pmol/mL.

12. A product for use according to claim 11 wherein dose of hCG to be administered to the patient is from 5,000 IU to 10,000 IU.

13. A product for use according to any of claims 6 to 12 wherein the use comprises a further step of administering a GnRH antagonist starting on day 6 of stimulation.

14. A product for use according to any preceding claim wherein the use further comprises: retrieving (e.g. harvesting) oocyte(s); fertilizing (e.g. inseminating) the oocytes(s); allowing the fertilized oocytes to develop to the blastocyst stage; and freezing one or more (or all) blastocysts.

15. A product for use according to any preceding claim wherein the menotropin and a rFSH including α2,3- and α2,6-sialylation are administered separately, for example the rFSH including α2,3- and α2,6-sialylation is for administration in the morning and the menotropin is for administration in the afternoon or evening.

16. A product for use according to any preceding claim wherein the menotropin and a rFSH including α2,3- and α2,6-sialylation are administered together.

17. A product for use according to any preceding claim wherein the menotropin is highly purified menotropin (HP-hMG).

18. A product for use according to any preceding claim wherein the rFSH is an rFSH wherein 50 to 95% of the total sialylation is α2,3-sialylation and wherein 5-50% of the total sialylation is α2,6-sialylation.

19. A product for use according to any preceding claim wherein the daily dose of menotropin (human menopausal gonadotropin or hMG) is 75 IU when the recombinant human follicle stimulating hormone (rFSH) including α2,3- and α2,6-sialylation daily dose is <12 μg; 150 IU when the recombinant human follicle stimulating hormone (rFSH) including α2,3- and α2,6-sialylation daily dose is 12 μg and patient body weight is <100 kg; and 225 IU when the recombinant human follicle stimulating hormone (rFSH) including α2,3- and α2,6-sialylation daily dose is 12 μg and patient body weight is ≥100 kg.

20. A product for use according to any preceding claim wherein the use comprises a further step of assessing follicular growth by ultrasonography e.g., on day 9, 10, 11, 12, 13, 14 etc. of stimulation.

21. A method of treatment of infertility in a patient of age≥35 (e.g., age 35 to 40, e.g., age 35-36) and having a serum AMH level of <15 pmol/L prior to treatment, comprising;
identifying a patient of age≥35 (e.g., age 35 to 40, e.g., age 35-36) and having a serum AMH level of <15 pmol/L prior to treatment; and
administering to the patient human menopausal gonadotropin (hMG) and a recombinant human follicle stimulating hormone (rFSH) including α2,3- and α2,6-sialylation, wherein the rFSH is administered at a dose a dose of, or equivalent to, 11-13 μg (e.g., 12 μg) per day of the rFSH, and the hMG is administered at a dose of, or equivalent to, 37.5 IU to 225 IU per day of highly purified hMG (HP-hMG).

22. A method of treatment of infertility in a patient of age≥35 (e.g., age 35 to 40, e.g., age 35-36) and having a serum AMH level of <15 pmol/L prior to treatment, comprising;
identifying a patient of age≥35 (e.g., age 35 to 40, e.g., age 35-36) and having a serum AMH level of ≥15 pmol/L prior to treatment; and
administering to the patient human menopausal gonadotropin (hMG) and a recombinant human follicle stimulating hormone (rFSH) including α2,3- and α2,6-sialylation, wherein the rFSH is administered at a dose a dose of, or equivalent to, 0.09-0.19 μg of the rFSH per kg body weight of the patient per day, and the hMG is administered at a dose of, or equivalent to, 37.5 IU to 225 IU per day of highly purified hMG (HP-hMG).

23. A pharmaceutical composition comprising a menotropin (human menopausal gonadotropin or hMG) for use in the treatment of infertility in a patient of age≥35 (e.g., age 35 to 40, e.g., age 35-36) having a serum AMH level of <15 pmol/L prior to treatment/stimulation; wherein the pharmaceutical composition is to be administered at a dose of, or equivalent to, 37.5 IU to 225 IU HP-hMG per day of the menotropin, together with a dose of, or equivalent to, 11-13 μg (e.g., 12 μg) per day of a recombinant human FSH (rFSH) including α2,3- and α2,6-sialylation.

24. A pharmaceutical composition comprising a recombinant human follicle stimulating hormone (rFSH) including α2,3- and α2,6-sialylation for use in the treatment of infertility in a patient of age≥35 (e.g., age 35 to 40, e.g., age 35-36) having a serum AMH level of <15 pmol/L prior to treatment/stimulation; wherein the pharmaceutical composition is to be administered at a dose of, or equivalent to, 11-13 μg (e.g., 12 μg) per day of the recombinant human FSH including α2,3- and α2,6-sialylation, together with a dose of, or equivalent to, 37.5 IU to 225 IU HP-hMG per day of a menotropin (human menopausal gonadotropin or hMG).

25. A pharmaceutical composition comprising a menotropin (human menopausal gonadotropin or hMG) for use in the treatment of infertility in a patient of age≥35 (e.g., age 35 to 40, e.g., age 35-36) having a serum AMH level of ≥15 pmol/L prior to treatment/stimulation; wherein the pharmaceutical composition is to be administered at a dose of, or equivalent to, 37.5 IU to 225 IU HP-hMG per day of the menotropin, together with a dose of, or equivalent to, 0.09-0.19 μg per kg body weight of the patient per day of a recombinant human FSH including α2,3- and α2,6-sialylation.

26. A pharmaceutical composition comprising a recombinant human follicle stimulating hormone (rFSH) including α2,3- and α2,6-sialylation for use in the treatment of infertility in a patient of age≥35 (e.g., age 35 to 40, e.g., age 35-36) having a serum AMH level of ≥15 pmol/L prior to treatment/stimulation; wherein the pharmaceutical composition is to be administered at a dose of, or equivalent to, 0.09-0.19 μg per kg body weight of the patient per day of the recombinant human FSH including α2,3- and α2,6-sialylation, together with a dose of, or equivalent to, 37.5 IU to 225 IU HP-hMG per day of the menotropin (human menopausal gonadotropin or hMG).
